(19) United States
(12) Patent Application Publication
(10) Pub. No.: US 2008/0038219 A1
(43) Pub. Date: Feb. 14, 2008

(54) Novel Composition for a Topical Skin Treatment Base and Medicated Applications Thereof

(75) Inventors: James D. Mosbaugh, Tampa, FL (US); Grant D. Carlson, Lakeland, FL (US)

Correspondence Address: JIM MOSBAUGH;CALGENEX CORPORATION, 9950 PRINCESS PALM AVENUE, SUITE 320, TAMPA, FL, 33619, US

(73) Assignee: CALGENEX CORPORATION, Tampa, FL (US)

(21) Appl. No.: 11/835,213

(22) Filed: Aug. 7, 2007

Related U.S. Application Data
(60) Provisional application No. 60/821,674, filed on Aug. 7, 2006.

(51) Int. Cl. A61K 8/97; A61K 31/7034; A61K 31/63; A61K 31/43; A61K 31/545; A61K 36/28; A61K 36/48

(52) U.S. Cl. 424/74; 514/35; 514/192; 514/155; 514/200; 514/57; 424/764; 424/757

(57) ABSTRACT

A novel topical preparation comprised of carboxylic acids, chelating agents, dimethyl Sulfone and magnesium sulfate that forms a functional and versatile base formation for the addition of numerous medications and active ingredients for the purpose of treating certain skin conditions including, but not limited to psoriasis, eczema, dermatitis, acne, rosacea, scleroderma, skin stones, fungal infections, bacterial infections, or other skin disorders and diseases with improved efficacy and penetration

This application claims the benefit of Provisional Application No. 60/821,674 filed Aug. 7, 2006.

### FIELD OF THE INVENTION

The present invention relates to a topical anti-itch composition for applying to the skin of a patient to treat psoriasis, eczema and other skin disorders and to a new carrier formulation for the topical delivery of medicaments and other active ingredients to be used in prescription drugs, over the counter drugs, and cosmetics containing active ingredients (cosmeceuticals).

### BACKGROUND OF THE INVENTION

Topical skin products are ubiquitous to the personal care, over the counter drug (OTC) and prescription drug markets. The delivery of drugs or other active ingredients is accomplished by placing the desired drug or active compound in a base that is typically a lotion, cream, gel, ointment, or other solution or suspension. Presently, there is a new trend in the personal care and topical drug industries (both OTC and prescription) wherein the term “cosmeceuticals” has become accepted as the blend between active ingredients and more standard products. There are many over the counter (OTC) and prescription products that are sold in the form of a lotion or gel or similar for the transdermal application of drugs or other active ingredients. Much research has focused on safely enhancing the absorption of said products as well as simultaneously providing therapeutic skin conditioners.

Skin disorders, as the term is used herein, encompasses numerous skin conditions ranging in severity from severe dermatitis, severe dry skin, psoriasis, bacterial infections, fungal infections, acne, rosacea, scleroderma, skin stones etc., to less severe conditions, such as lack of adequate skin firmness, dermal hydration or sebum secretion, etc., which are nonetheless unsightly and may cause physical discomfort.

The skin disorder Psoriasis is a non-contagious, lifelong skin disease that has been diagnosed in 4.5 million adults in the United States. The most common form, plaque psoriasis, appears as raised, red patches or lesions covered with a silvery white buildup of dead skin cells, called scale. Psoriasis is a very diverse skin disease that appears in a variety of forms. Each form has distinct characteristics. Typically, people have only one type of psoriasis at a time, but occasionally two or more different types of psoriasis can occur at the same time. Psoriasis can also occasionally change from one form to another. Trigger factors may “convert” some forms of psoriasis, such as plaque, to another form, such as pustular. Generally, one type of psoriasis will clear and then another form of psoriasis will appear later.

Plaque psoriasis is the most prevalent form of the disease. About 80 percent of all those who have psoriasis have this form. Its scientific name is psoriasis vulgaris (vulgaris means common). It is characterized by raised, inflamed, red lesions covered by a silvery white scale. It is typically found on the elbows, knees, scalp and lower back.

Guttate psoriasis is a form of psoriasis that often starts in childhood or young adulthood. The word guttate is from the Latin word meaning “drop.” This form of psoriasis resembles small, red, individual spots on the skin. Guttate lesions usually appear on the trunk and limbs. These spots are not usually as thick as plaque lesions. Guttate psoriasis often comes on quite suddenly. A variety of conditions have been known to bring on an attack of guttate psoriasis, including upper respiratory infections, streptococcal infections, tonsillitis, stress, injury to the skin and the administration of certain drugs (including antimalarials and beta-blockers). A streptococcal infection of the throat (strep throat) is a common guttate psoriasis trigger. Strep throat can be present without symptoms and can still cause a flare of guttate psoriasis. Talk with your doctor about getting a strep test to determine if you have an underlying strep infection. Guttate psoriasis may persist despite clearance of the strep infection. Some doctors prescribe antibiotics to help prevent an occurrence of an infection that can cause the outbreak of guttate psoriasis. Moisturizers are considered the preferred treatment for guttate psoriasis. However, people often find it tedious to apply topical preparations to the multiple small “drops” on their skin. Phototherapy treatment with ultraviolet light B (UVB) or PUVA (the light-sensitizing drug psoralen plus ultraviolet light A) is also very effective for guttate psoriasis. Only in severe cases will doctors prescribe systemic medications (drugs given orally or by injection) for this type of psoriasis, although sometimes a short course of one of these drugs results in rapid and prolonged clearing.

Inverse psoriasis is found in the armpits, groin, under the breasts, and in other skin folds around the genitals and the buttocks. This type of psoriasis first shows up as lesions that are very red and usually lack the scale associated with plaque psoriasis. It may appear smooth and shiny. Inverse psoriasis is particularly subject to irritation from rubbing and sweating because of its location in skin folds and tender areas. It is more common and troublesome in overweight people and people with deep skin folds. Treatment can be difficult due to the sensitivity of skin in these areas. Steroid creams and ointments are considered very effective, but they should not be occluded (covered) with plastic dressings. Overuse or misuse of steroids, particularly in skin folds, can result in side effects, especially thinning of the skin and stretch marks. Because these areas are prone to yeast and fungal infections, doctors may test for infection and then may use diluted topical steroids in combination with other medications, for example, 1% or 2% hydrocortisone with anti-yeast or antifungal agents. Other topical agents, such as Dovonex, coal tar or anthralin, can be somewhat effective in treating psoriasis in skin folds, but they may also be irritating. They should be used with caution and under the direction of a doctor. People with severe inverse psoriasis may occasionally require systemic medications to control the condition. Protopic (also known by its generic name tacrolimus) and Elidel (also known by its generic name pimecrolimus) are two topical medications approved by the U.S. Food and Drug Administration for the treatment of eczema. Many dermatologists have found they work well for psoriasis lesions in the skin folds.

Pustular psoriasis, primarily seen in adults, is characterized by white pustules (blisters of noninfectious pus) surrounded by red skin. The pus consists of white blood cells. It is not an infection, nor is it contagious. It may be localized to certain areas of the body—for example, the hands and feet. Pustular psoriasis also can be generalized, covering most of the body. It tends to go in a cycle-reddening of the skin followed by formation of pustules and scaling. Pustular psoriasis reportedly may be triggered by internal medications, irritating topical agents, overexposure to UV light, pregnancy, systemic steroids, infections, emotional stress and sudden withdrawal of systemic medications or potent topical steroids. It is not unusual for doctors to combine or rotate treatments for pustular psoriasis due to the potential side effects of systemic medications and phototherapy. More than one study has shown that Soriatane (also known by its generic name acitretin) and methotrexate in combination produced a rapid remission in the acute state of pustular psoriasis and an eventual clearing of the skin.

Erythrodermic psoriasis is a particularly inflammatory form of psoriasis that often affects most of the body surface. It may occur in association with von Zumbusch pustular psoriasis. It is characterized by periodic, widespread, fiery redness of the skin. The erythema (reddening) and exfoliation (shedding) of the skin are often accompanied by severe itching and pain. Patients having an erythrodermic psoriasis flare should make an appointment to see a doctor immediately. Erythrodermic psoriasis causes protein and fluid loss that can lead to severe illness. Edema (swelling from fluid retention), especially around the ankles, may also develop along with infection. The body's temperature regulation is often disrupted, producing shivering episodes. Infection, pneumonia and congestive heart failure brought on by erythrodermic psoriasis can be life threatening. People with severe cases of this condition often require hospitalization. Known triggers of erythodermic psoriasis include abrupt withdrawal of systemic treatment; the use of systemic steroids (cortisone); an allergic, drug-induced rash that brings on the Koebner response (a tendency for psoriasis to appear on the site of skin injuries); and severe sunburns.

Initial treatment usually includes medium-potency topical steroids and moisturizers, combined with wet dressings, oatmeal baths and bed rest. Antibiotics may also be used. Careful attention is paid to restoring and maintaining fluids in the body. In addition, methotrexate, Soriatane or cyclosporine are frequently required to bring severe cases under control. Use of systemic steroids for erythrodermic psoriasis is controversial, and if used, they should be tapered off slowly. Stopping them suddenly can trigger a flare of psoriasis. UVB or PUVA treatment is usually held in reserve until the degree of redness has improved.

Eczema, or dermatitis as it is sometimes called, is a group of skin conditions which can affect all age groups. Up to one fifth of all children of school age have eczema, along with about one in twelve of the adult population. The severity of the disease can vary. In mild forms the skin is dry, hot and itchy, whilst in more severe forms the skin can become broken, raw and bleeding. Although it can sometimes look unpleasant, eczema is not contagious. With treatment the inflammation of eczema can be reduced, though the skin will always be sensitive to flare-ups and need extra care. The causes of eczema are many and varied, and depend on the particular type of eczema that a person has. Atopic eczema is thought to be a hereditary condition, being genetically linked. It is proposed that people with atopic eczema are sensitive to allergens in the environment which are harmless to others. In atopy there is an excessive reaction by the immune system producing inflamed, irritated and sore skin. Associated atopic conditions include asthma and hay fever. Other types of eczema are caused by irritants such as chemicals and detergents, allergens such as nickel, and yeast growths. In later years eczema can be caused by blood circulatory problems in the legs. The causes of certain types of eczema remain to be explained, though links with environmental factors and stress are being explored. There are several different types of eczema, many of which look similar but have very different causes and treatments. The first step in effective treatment of eczema is a correct diagnosis. It is very important to see a general practitioner in the first instance, who may make a referral to a specialist dermatologist for further diagnosis and treatment.

Atopic eczema is the commonest form of eczema and is closely linked with asthma and hay fever. It can affect both children and adults, usually running in families. One of the most common symptoms of atopic eczema is its itchiness (or pruritis), which can be almost unbearable. Other symptoms include overall dryness of the skin, redness and inflammation. Constant scratching can also cause the skin to split, leaving it prone to infection. In infected eczema the skin may crack and weep (‘wet’ eczema). Treatments include emollients to maintain skin hydration and steroids to reduce inflammation.

Allergic contact dermatitis develops when the body's immune system reacts against a substance in contact with the skin. The allergic reaction often develops over a period of time through repeated contact with the substance. For example, an allergic reaction may occur to nickel, which is often found in earrings, belt buckles and jeans buttons. Reactions can also occur after contact with other substances such as perfumes and rubber. In order to prevent repeated reactions it is best to prevent contact with anything that you know causes a rash.

Irritant contact dermatitis this is a type of eczema caused by frequent contact with everyday substances, such as detergents and chemicals, which are irritating to the skin. It most commonly occurs on the hands of adults and can be prevented by avoiding the irritants and keeping the skin moisturized.

Infantile seborrhoeic eczema is a common condition affecting babies under one year old, the exact cause of which is unknown. Also referred to as cradle cap, it usually starts on the scalp or the nappy area and quickly spreads. Although this type of eczema looks unpleasant, it is not sore or itchy and does not cause the baby to feel uncomfortable or unwell. Normally this type of eczema will clear in just a few months, though the use of moisturizing creams and bath oils can help to speed this along.

Adult seborrheic eczema characteristically affects adults between the ages of 20 and 40. It is usually seen on the scalp as mild dandruff, but can spread to the face, ears and chest. The skin becomes red, inflamed and starts to flake. The condition is believed to be caused by a yeast growth. If the condition becomes infected, treatment with an anti-fungal cream may be necessary.

Varicose eczema affects the lower legs of those in their middle to late years, being caused by poor circulation. Commonly the skin around the ankles is affected, becoming speckled, itchy and inflamed. Treatment is with emollients and steroid creams. If left untreated, the skin can break down, resulting in an ulcer.

Discoid eczema is usually found in adults and appears suddenly as a few coin shaped areas of red skin, normally on the trunk or lower legs. They become itchy and can weep fluid. Usually discoid eczema is treated with emollients (and steroid creams if necessary).

There are a number of ways to manage eczema, all of which begin with an effective skin care routine. Having access to accurate information is important as this allows the person with eczema, or their care giver, to make informed choices when managing the condition. The following are the more commonly used treatments. Further information on any of these can be obtained through the National Eczema Society.

Emollients are necessary to reduce water loss from the skin, preventing the dryness normally associated with eczema. By providing a seal or barrier, the skin is less dry, itchy and more comfortable. Emollients are safe to use as often as is necessary and are available in various forms: ointments for very dry skin, creams and lotions for mild to moderate or ‘wet’ eczema. Some are applied directly to the skin, whilst others are used as soap substitutes or can be added to the bath. The range of emollients available is enormous and it may be necessary to try several before the most suitable one is found. Testing a small amount on the skin first is advisable, as emollients contain substances to which some people are sensitive.

Topical steroids. When eczema is under control, only emollients need to be used. However in flare-ups, when the skin becomes inflamed, a steroid cream may be needed. Steroids act by reducing inflammation and are used in most types of eczema. Topical steroids come in four different strengths, mild, moderately potent, potent and very potent. The strength of steroid cream that a doctor prescribes depends on the age of the patient, the severity of the condition and, the size of the area and part of the body to be treated. Topical steroids are applied thinly to the affected area, as directed by the prescribing doctor. Your eczema should be reviewed regularly if topical steroids are being applied. It is important to use only the steroid cream prescribed for yourself and not to lend or borrow (what may be) an unsuitable cream from someone else. Many people have concerns regarding the use of topical steroids and their side-effects. As long as steroids are used appropriately and as directed by your doctor, the likelihood of side effects is very rare. Reported side-effects have been largely due to the use of very potent steroid preparations over long periods of time.

Acne vulgaris is the most common of all skin disorders. It is a chronic inflammatory process that affects the pilosebaceous unit in virtually every adolescent and in many adults and prepubertal children as well. As such, it carries with it a heavy emotional and psychological burden.[1] Marion Sulzberger, MD, one of the founding figures of modern dermatology, wrote in 1948 that “there is no single disease which causes more psychic trauma, nor maladjustment between parents and children, more general insecurity and feelings of inferiority and general sums of psychic suffering than does acne vulgaris.”[2] The impact can be devastating, leading even to thoughts of suicide. But the impact is not entirely psychological. Research by William Cunliffe, MD, in the United Kingdom, showed that patients with acne had a higher unemployment rate than age- and sex-matched controls.[3]

Dr. Cunliffe also has studied the prevalence of facial acne in adults. A 1999 community-based study^{[4]} showed that 12% of women over the age of 25 had clinical acne, and acne prevalence did not significantly decrease until after age 44. These figures agree with the clinical experience of many physicians, who report seeing increasing numbers of adult acne patients—especially women—in their practices.

Acne vulgaris evolves within the pilosebaceous unit (FIG. 1) via a multifactorial pathogenesis. The central pathogenic factors in acne are^{[5,6]}:
Excessive sebum production secondary to androgen stimulation;
Abnormal follicular keratinization resulting in follicular plugging;
Proliferation of Propionibacterium acnes (P acnes), an anaerobic organism normally resident in the follicle; and
Inflammation following chemotaxis and the release of various proinflammatory mediators.

The increase in adrenal androgens during the prepubertal period triggers the enlargement of the sebaceous glands. These enlarged sebaceous glands produce increased amounts of sebum, which flows through the canal of the sebaceous follicle. This canal is lined with a keratinizing epithelium. In acne patients, there is increased production of the follicular corneocytes lining the follicle and retention of these corneocytes within the follicle. The abnormally desquamated corneocytes and the excess sebum build up within the follicle to form a microscopic, bulging mass. This enclosed, sebum-rich environment is ideal for the proliferation of P acnes, the anaerobic bacterium that produces chemotactic factors and recruits proinflammatory molecules involved in the inflammatory phase of acne.

Inflammatory acne lesions include papules, pustules, nodules, and cysts. A papule is a pink-to-red, raised, palpable lesion with no visible accumulation of fluid, which can range from 1 to 4 mm in diameter.

A pustule is a raised accumulation of purulent material on the skin's surface, and is similar in size to the papule. Pustules are sometimes characterized as superficial or deep. In a superficial pustule there is a localized rupture of the epithelium near the skin surface, and in a deep pustule there is extensive destruction of the entire epithelium. A nodule is a tender, firm lesion that may persist for weeks. Cysts may be as large as several centimeters in diameter, and they may drain a creamy, yellowish material. Darkly pigmented skin affected by acne tends to develop significant postinflammatory hyperpigmentation. This tendency has given rise to the suggestion that a new acne lesion should be designated^{[7]}—the acne hyperpigmented macule (AHM). The AHM can last for 4 months or longer, and is often the central complaint of acne patients with skin of color.

Topical therapy for acne can be divided into topical antimicrobials/antibiotics, topical retinoids, and miscellaneous topical therapy. Benzoyl peroxide (BP) is an antimicrobial that is very effective for killing P acnes. Products containing BP are widely available by prescription and over the counter. BP, however, has minimal impact on microcomedo formation and is generally best used in combination with topical retinoids (see below). BP-containing products are available in a variety of formulations, including gels, creams, lotions, washes, and bar soaps, in a variety of concentrations (most often 2.5%, 5%, and 10%). Concentration should be adjusted to skin type and tolerance, since BP may cause skin irritation, erythema, and dryness. Patients also should be advised that BP will bleach colored fabrics.

Topical antibiotics kill P acnes and also exhibit significant anti-inflammatory properties. Thus, while topical antibiotics do not have a major effect on comedo formation, they are active against inflammatory lesions such as papules and pustules. Because, like BP, these drugs do not have a major impact on comedo formation, they are most often used in combination with topical retinoids (see below). The most widely prescribed topical antibiotics are erythromycin and clindamycin. Topical clindamycin 1% and topical erythromycin 1.5% have been shown in a double-blind, randomized trial to be clinically equivalent in the treatment of moderate facial acne,^{[15]} and it is estimated that topical clindamycin is approximately equivalent to a 500-mg dose of tetracycline.^{[16]} Topical erythromycin and clindamycin were originally available as hydroalcoholic solutions dispensed in applicator bottles. Hydrophilic gels and lotions were developed in an attempt to reduce irritation and enhance patient convenience and compliance. More recently, the pledget delivery system has become widely used. Erythromycin is also available in a creamy ointment formulation (Akne-Mycin).

Newer products combine topical BP and either erythromycin (3% erythromycin, 5% BP; Benzamycin) or clindamycin (1% clindamycin, 5% BP; BenzaClin, Duac) in gel vehicles. Clinical studies enrolling patients with mild to moderately severe acne have demonstrated the increased efficacy of the combination products compared with either agent alone, without increased side effects.^{[17,18]} The emergence of antibiotic-resistant P acnes is an issue of increasing concern with both topical and oral antibiotics in the treatment of acne.^{[19,21]} Over the past 25 years, laboratory studies have demonstrated a rapidly increasing pattern of P acnes resistance to antibiotics, especially erythromycin. This seems to be less likely with clindamycin. For this reason, it seems prudent to minimize the use of topical and/or oral antibiotics in acne, replacing them as soon as possible with BP and/or topical retinoids. Combination therapy with topical antibiotics and BP or the use of a combination product containing BP and erythromycin or clindamycin may also prevent the emergence of drug-resistant P acnes.^{[22,23]}

The topical retinoids include vitamin A acid (tretinoin), its analogs, and newer agents that bind to and activate retinoid receptors. Topical retinoids are the treatment of choice for comedonal acne and are definitely the most effective agents for clearing microcomedones. Initially, topical retinoids were prescribed only for comedonal acne. Today, however, there is strong evidence that topical retinoids have both direct and indirect anti-inflammatory actions,^{[24]} and their use in inflammatory acne is expanding rapidly. The emerging consensus among acne experts is that topical retinoids are a front-line therapy for inflammatory acne and should be used early in treatment.^{[25]} Topical retinoids seem to be especially effective in combination therapy with BP or topical antibiotics. Such combination therapy seems quite logical when one recalls the basic pathophysiology of acne, ie, a combination of comedo formation, proliferation of P acnes, and inflammation. Thus, the value of a regimen that combines antibiotics to kill P acnes and suppress inflammation with topical retinoids to resolve comedones and add additional anti-inflammatory effects is clear. Another, newer concept in the use of topical retinoids is maintenance therapy.^{[25]} Studies have shown that after acne has resolved clinically, microcomedones (the primary lesions in acne) begin to recur. Thus, it would seem reasonable to use topical retinoids in virtually all acne patients who have cleared in order to prevent or reduce recurrence.

The major problem in using topical retinoids has been irritation. Irritation, strongly associated with the earlier retinoids, has led to 2 significant clinical misconceptions. The first misconception is that irritation might be necessary for retinoids to achieve therapeutic success. The emergence of less irritating but very effective topical retinoids (see below) has disproved this notion. Cutaneous irritation is clearly an undesirable side effect of retinoid use and may be attenuated by using a product with low irritancy potential and carefully instructing patients in proper skin care (wash gently with tepid water and mild soaps or soap substitutes; avoid washcloths and astringents; use sunscreens; and avoid excessive sun exposure). The second misconception is that retinoids should not be used in inflammatory acne. Several studies now show that retinoids possess direct and indirect anti-inflammatory properties^{[24]} and should be first-line therapy for inflammatory acne. Another concern is the risk of teratogenicity with topical retinoids. Tazarotene, for example (see below), is a pregnancy category X drug. Since only minute amounts of topically applied retinoids ever reach the bloodstream, such a risk seems remote. Nevertheless, it would be prudent to avoid the use of topical retinoids in pregnant women. Irritation was a particular problem with the first-generation pioneering topical retinoid tretinoin. Newer formulations of tretinoin aim to reduce irritation through the use of vehicles that slow release of the active agent. One such product (Avita), available in 0.025% gel or cream vehicles, uses polymer compounds to slow release of the tretinoin and reduce irritation.^{[26]} Another (Retin-A Micro) slows drug delivery and decreases irritation by incorporating the active ingredient into microsponges.

A newer generation of receptor-selective retinoids, such as adapalene (Differin) and tazarotene (Tazorac), may have certain therapeutic advantages over tretinoin. Adapalene is a third-generation retinoid that selectively targets retinoic acid receptors found primarily in the epidermis. The drug modulates cellular differentiation, keratinization, and inflammatory processes.^{[27]} Adapalene is available in 0.1% gel, solution, cream, and pledget formulations.

In an investigator-masked, randomized, parallel-group, multicenter trial enrolling patients with mild-to-moderate acne, adapalene gel 0.1% was significantly more effective at reducing inflammatory and noninflammatory lesions than was tretinoin gel 0.025%.^{[28]} Adapalene gel 0.1% has been found to be as effective as tretinoin cream 0.05%.^{[29]} The most common adverse effects associated with adapalene treatment are erythema, scaling, dryness, pruritus, and burning. In comparative trials,^{[28,29]} adapalene gel was better tolerated than tretinoin 0.025% gel and 0.05% cream. In tests of cumulative irritation potential in healthy subjects,^{[30]} adapalene gel 0.1% was better tolerated than tretinoin 0.1%, 0.05%, and 0.025% creams; tretinoin 0.025% and 0.01% gels; and tretinoin 0.1% gel microsphere. Tazarotene, a synthetic acetylenic retinoid, is another third-generation receptor-selective retinoid. The drug modulates cellular differentiation, proliferation, and the inflammatory process. It is available in 0.1% cream and gel formulations for the treatment of acne.

In a double-blind, multicenter, randomized, parallel-group trial^{[31]} enrolling patients with mild-to-moderate acne, tazarotene 0.1% gel was more effective than 0.025% tretinoin gel in reducing noninflammatory lesions and as effective in reducing inflammatory lesions. Tazarotene gel was also found to be more effective than tretinoin 0.1% microsponge gel as measured by overall disease severity and noninflammatory lesion count.^{[32]} A double-blind, randomized, parallel-group trial found tazarotene 0.1% gel to be more effective than adapalene 0.1% gel in the treatment of mild-to-moderate acne.^{[33]}

Older therapies still available include various products containing sulfur, resorcinol, and salicylic acid. These are generally less effective than the newer topical agents discussed above. A relatively new agent is azelaic acid (Azelex), a dicarboxylic acid available in a 20% cream with efficacy against inflammatory (and to a lesser degree, comedonal) lesions. Azelaic acid may also be useful in treating cutaneous hyperpigmentation, including acne-induced postinflammatory hyperpigmentation. In clinical trials, treatment of mild-to-moderate acne with azelaic acid has shown efficacy comparable to that of tretinoin 0.05%, benzoyl peroxide 5%, and topical erythromycin 2%.^{[35]} The most frequent adverse reactions are pruritus, burning, stinging, and tingling.

It has therefore been shown the hydrocortisone is a common, safe, and accepted treatment for psoriasis, eczema, acne, and other skin disorders in the form of a topical cream or lotion. Hydrocortisone is a steroid hormone secreted by the adrenal cortex. Commercially, it is available as the unchanged hormone and as hydrocortisone acetate, hydrocortisone cypionate, hydrocortisone sodium phosphate, hydrocortisone butyrate, hydrocortisone valerate, and hydrocortisone sodium succinate. Hydrocortisone is the preferred glucocorticoid for replacement therapy in patients with adrenal insufficiency, although some patients require concomitant administration of a more potent mineralocorticoid, such as fludrocortisone, to treat this condition. Topical hydrocortisone is considered low potency. Low potency topical corticosteroids are the safest for chronic use and may be used on the face or intertriginous areas, with occlusion, and in infants and young children. Hydrocortisone was approved by the FDA in 1951.

Endogenous corticosteroids are secreted by the adrenal cortex, and their effects are believed to be due to enzyme modification rather than to a direct hormone-induced action. Corticosteroids are loosely classified into two categories, mineralocorticoids and glucocorticoids, depending on their primary pharmacological activity. Mineralocorticoids alter electrolyte and fluid balance by facilitating sodium resorption and hydrogen and potassium excretion at the level of the distal renal tubule, resulting in edema and hypertension. Glucocorticoids exert some mineralocorticoid effects but are also involved in a number of other metabolic pathways including gluconeogenesis, fat redistribution, protein metabolism, and calcium balance. Hydrocortisone possesses both mineralocorticoid actions and glucocorticoid actions.

Corticosteroids exhibit anti-inflammatory, antipruritic, and vasoconstrictive properties. At the cellular level, corticosteroids induce peptides called lipocortins. Lipocortins antagonize phospholipase A₂, an enzyme which causes the breakdown of leukocyte lysosomal membranes to release arachidonic acid. This action decreases the subsequent formation and release of endogenous inflammatory mediators including prostaglandins, kinins, histamine, liposomal enzymes and the complement system. Topical preparations of hydrocortisone are metabolized in the skin, while systemic hydrocortisone is metabolized by the liver to inactive metabolites. These inactive metabolites, as well as a small portion of unchanged drug, are excreted in the urine. The biological half-life of hydrocortisone is 8-12 hours.

Coal tar is indicated for the symptomatic management of pruritus and irritation caused by dandruff, seborrheic dermatitis, atopic dermatitis, eczema, and psoriasis. Treatment with coal tar and UV light or sunlight can be beneficial because of its photosensitizing action. Official USP coal tar preparations include crude coal tar, coal tar topical solution, and coal tar ointment. Crude coal tar is produced as a byproduct secondary to the destructive distillation of coal, and it can be further refined into coal tar topical solution or ointment. Commercially available preparations that are not officially recognized by USP and do not have specifications for composition include coal tar extract and distillate. Application of crude coal tar preparations may be aesthetically displeasing to the patient; however, further refined products are believed by many clinicians to be therapeutically inferior. It has been suggested that the variability in refining processes may be responsible for differences in therapeutic response to coal tar preparations. Coal tar was in use prior to 1938 and approved by the FDA at its inception.

Coal tar exhibits keratoplastic and mild irritant activity. Coal tar may decrease the quantity and size of epidermal cells produced and inhibit mitosis, possibly through removal of oxygen in the skin. Shampoo and soap preparations may exert their action through absorption into the epidermis and enhancement of scale removal. It has been suggested that a reaction similar to that following exposure to sunlight can occur in the epidermis through interaction between the peroxides in coal tar and epidermal sulfhydryl groups. Subsequently, epidermal proliferation may be decreased. Coal tar preparations are also believed to possess antipruritic, antiseptic, astringent, antifungal, vasoconstrictive, and photosensitizing properties. Coal tar is reportedly carcinogenic in humans, inducing skin cancer primarily in the anogenital region, following prolonged exposure to coal tar in industrial settings. It is unlikely that patients treated acutely for dermatologic conditions are at an increased risk for developing skin cancer. Nevertheless, this risk should be considered during prolonged treatment periods.

Most coal tar preparations used for dermatologic disorders contain 2-5% coal tar. Coal tar is applied topically in various formulations such as creams, gels, ointments, bath preparations, shampoos, liquid preparations (lotions and emulsions), and cleansing bars and solutions. The location and type of lesion will determine the appropriate formulation. It is unknown if coal tar preparations pose a fetal risk or if they are distributed into breast milk (see Contraindications). Coal tar and salicylic acid are used together in a topical preparation to treat eczema, psoriasis, and seborrheic dermatitis. Coal tar has keratoplastic action as well as antipruritic and anti-eczematous actions. Salicylic acid is added for its keratolytic activity.

Many such products exist presently in both prescription and over the counter drug forms. The most common over the counter form of hydrocortisone is 0.25 to 1.00% by weight creams comprised of a viscous fatty acid carrier and the drug. Some of these products incorporate various emollients, moisturizers, pH modifiers, emulsifiers, preservatives, or other excipients. Many times, the base used to supply the active ingredient is chosen partly in order to adequately solubilize or emulsify the desired drug or actives such that said actives are stable in the product formulation and do not interact with the other product components during manufacture, storage, or application. Ingredients for the preparation of stable lotions, gels, etc. include polymers that provide the substantive body or viscosity of the preparation, emollients that provide good skin feel, moisturizers that help to keep water on the surface of the skin, emulsifiers that keep all the ingredients in one homogeneous state, preservatives that keep the product from going rancid with microbial growth, chelating agents and antioxidants that scavenge free radicals and heavy metals that can reduce viscosity and cause an undesirably appearance and/or smell, and fragrance or colorants that enhance the appearance and smell of the product.

Traditional topical creams and lotions that contain one or more active or drug are comprised of waxy fatty acids that are melted and neutralized that provide a creamy base for the addition and emulsification of other ingredients. Stearic and palmitic acid are two common cream bases that used in products such as anti-itch creams or psoriasis treatments like coal tar and salicylic acid preparations. Recently, manufacturers have begun to add skin conditioning ingredients such as aloe or vitamin E to these products as well.

Many of the products that treat symptoms or indications of skin disorders come in a cream carrier that is difficult to spread, especially over damaged, sensitive skin. Additionally, many of the existing products reply on the drug or active to manage the symptoms or indications of the skin disorder. These products build the lotion base to accommodate the medicament, typically using stearic acid, palmitic acid, or petrolatum as the vehicle.

Several formulations have been proposed to overcome the disadvantages of the prior art, both for treating skin disorders, and for use in cosmetics in order to prevent skin irritation and clear blemishes.

U.S. Pat. No. 6,572,868, issued Jun. 3, 2003 to Sandra E. Cope, discloses a restructuring complex for cosmetic compositions. The composition comprises safe and effective amounts of carrageenans, borage seed oil, squalane, ceramide 3, ceramide 6, red algae extract, dipalmitoyl hydroxproline, and oleuropein.

U.S. Pat. No. 6,193,987, issued Feb. 27, 2001 to M. H. Harbeck, discloses a lubricating composition for the hands and skin. The composition has as its constituents a mixture of organic safflower oil, flaxseed oil, tincture of benzoin, and organic beeswax.

U.S. Pat. No. 6,479,043, issued Nov. 12, 2002 to Tietjen et al., discloses a depilatory composition. The composition includes emollients, skin conditioners, buffering agents, viscosity increasing agents, emulsion stabilizers, pH adjusters, chelating agents, fragrance, color, lubricants, propellants, or biological agents.

Other related patents include U.S. Pat. No. Re. 33,107, issued Nov. 7, 1989 to Dickstein et al. (compositions containing 1 .alpha.-hydroxycholecalciferol for topical treatment of skin disorders and methods employing same); U.S. Pat. No. 4,737,360, issued Apr. 12, 1988 to Allen et al. (skin care compositions comprising a pollen extract and non-animal and non-mineral oils); U.S. Pat. No. 5,350,774, issued Sep. 27, 1994 to C. Palou (therapeutic preparation for topical application to the skin); U.S. Pat. No. 5,824,323, issued Oct. 20, 1998 to Y. Fishman (skin lotion composition and softgel filled therewith and methods for making and using same); U.S. Pat. No. 5,916,573, issued Jun. 29, 1999 to Spiers et al. (topical treatment of the skin with a grape seed oil composition); U.S. Pat. No. 6,576,269, issued Jun. 10, 2003 to Korneyev (treating open skin lesions using extract of sea buckthorn); WO 01/37792, published May 31, 2001 (cosmetic skin care composition); and French Patent No. 2,806,906, published Oct. 5, 2001 (composition for use on the skin surrounding the eyes and mouth).

Various topical formulations and oral regimens of vitamins and herbs have been proposed for the treatment of skin conditions. U.S. Pat. No. 6,228,387, issued May 8, 2001 to M. Borod, describes a first composition for topical application and a second composition for oral administration for the treatment of hemorrhoids. The topical composition includes several herbs and vitamins, including grape seed extract and vitamin E, and in one embodiment, a few drops of Essential Oil of Chamomile.

U.S. Pat. No. 6,994,863 by Eini, et al. discloses Pharmaceutical and cosmetic carrier and composition for topical application a pharmaceutical or cosmetic carrier or composition for topical application characterized by rheological properties which render the carrier or composition semi-solid at rest and a liquid upon application of shear forces thereto. The composition or carrier are prepared by mixing 1 to 25 percent of a solidifying agent and 75 to 99 percent of a hydrophobic solvent, by weight, wherein at least one of them has therapeutic or cosmetic benefits, in the presence or absence of a biologically active substance.

U.S. Pat. No. 6,881,756 by Gendimenico discloses a method for treating skin disorders relating to a method for reducing inflammation in the skin and/or treating inflammatory skin disorders, pain, or pruritis by topically applying a composition comprising totarol or a pharmaceutically-acceptable ester thereof.

U.S. Pat. No. 6,461,699 by Ford discloses a cream carrier which has use as a cream-type carrier for topical delivery of medicaments including analgesics. The carrier comprises a mixture of: squalane NF, an emulsifier such as Tween 80, glycerin, cetyl alcohol NF, glyceryl monostearate, lecithin organogel preserved, BHT, urea USP, EDTA, water, stearic acid, simethicone USP, and ethoxy diglycol reagent. The invention also comprises a combination of the carrier, with either or both of ketamine hydrochloride and amitriptyline hydrochloride, which has use as a topically applied analgesic. Whereas Ford describes the use of EDTA, it is has limitation of use in a preservative manner and not for therapeutic value.

U.S. Pat. No. 6,399,093 by Petrus discloses a method and composition for the treatment of musculoskeletal disorders in mammals by the application of a topical composition comprising a permeation enhancing amount of one or more penetration enhancers, and one or more bio-affecting agents to provide anti-inflammatory relief and analgesia to the applied body part. Petrus claims the use of methyl sulfonyl methane, aloe, magnesium and antioxidants that is remotely similar to the present art, however Petrus describes that the formulation is meant to address musculoskeletal disorders such sprains, strains, tendinitis, tenosynovitis, fibromyalgia, osteoarthritis, rheumatoid arthritis, gout, pseudogout (calcium pyrophosphate deposition disease), polymyalgia rheumatica, bursitis, acute and chronic back pain and osteoporosis, which interfere with the normal performance of activities of daily living. Injuries include sprains, strains and tears of ligaments, tendons, muscles and cartilage damage. Petrus does not address skin conditions and his formulation wt % are substantially different from the present art.

U.S. Pat. No. 6,905,675 by Shackni et al. discloses a sulfur containing dermatological composition and methods for reducing malodors as caused by the sulfur. Shackni describes dermatological compositions wherein the pH is adjusted between about 6.5 and about 8.1. Shakni uses some of the ingredients as listed in the present art, however not at the specific weight percents or ratios as described in the present art.

None of the above inventions and patents, taken either singly or in combination, is seen to describe the instant invention as claimed. In particular, none of the above patents or publications has described a composition combining all of the naturally and synthetically occurring ingredients of the present skin formulation for topical application, and none have proven as effective as the present skin formulation for treatment of skin conditions such as dermatitis and psoriasis. Thus, a skin formulation solving the aforementioned problems is desired comprising exceptional lubricity, functionality of medicinal ingredients, low pH, and mineral balancing activity.

### SUMMARY OF THE INVENTION

The present invention relates to an improved composition for the delivery of topical medicaments for topical medications and therapeutic agents for cosmetic products and for treatment of skin conditions such as, but not limited to psoriasis, dermatitis, scleroderma, eczema, acne, or other inflammatory diseases of the skin. More specifically, the improved composition contains a novel combination of natural and synthetic ingredients provided specific ratios to one another including citric acid, magnesium sulfate, methyl sulfonyl methane, and disodium EDTA in a base that can accept a wide assortment of medicines including corticosteroids, coal tar, salicylic acid, benzoyl peroxide, camphor, antibiotics such a neomysin, tetracycline, bacitracin, anti-fungal agents and therapeutic cosmeceuticals ingredients including peptides, botanical extracts, aspartic acid, amino acids, etc.

### DETAILED DESCRIPTION OF THE INVENTION

The unique composition of the present invention comprises a novel blend of cationic species in conjunction with synthetic amino acid EDTA and anionic citrates.

In one aspect, the invention comprising a pharmaceutical carrier for topical application of medicaments consisting of a lotion or gel like base comprising a mixture of the following

Aloe Barbadensis Leaf Juice Powder or aloe vera leaf water (between 40-90%)

Butyrospermum Parkii (Shea Butter) (between 0.20 and 20%)

Caprylyl Glycol (between 0.1 and 2%)

Carbomer (between 0.40 and 2%)

Cellulose Gum (between 0.1 and 5%)

Chamomilla Recutita (Matricaria) Flower Extract (between 0.1 and 4%)

Citric Acid (between 0.15 and 5%)

Dimethicone Copolyol (between 0.20 and 10%)

Dimethyl Sulfone (MSM) (between 0.20 and 15%)

Disodium EDTA (between 0.10 and 3%)

Hexylene Glycol (between 0.05 and 1.0%)

Isopropyl Myristate (between 0.20 and 10%)

Magnesium Sulfate (Epson Salts) (between 0.10 and 10%)

Phenoxyethanol (between 0.10 and 1%)

Polysorbate 80 (between 0.50 and 3%)

Potassium Sorbate (between 0.2 and 2%)

Propylene Glycol (between 0.20 and 10%)

Simmondsia Chinensis (Jojoba) Seed Oil (between 0.20 and 15%)

Tocopherol or gamma/delta tocotrienol (between 0.15 and 2%)

Triethanolamine (between 0.25 and 5%)

Water (between 40 and 85%)

Furthermore, the ration of magnesium to citric acid ranges between 0.20 to 3.00, 0.50 to 2.00, more preferably 0.8 to 1.

Medicine or other active ingredients may be added to the formulation in oil or water phase from approximately 10-30% by weight.

The composition of the present invention is able to receive and hold with great stability many different medicaments or active cosmetic compounds. Depending upon the end use of the formulation, various medicines and/or cosmetic ingredients may be added into the oil or water water phase of the product prior to making the emulsion. These compounds, hereafter referred to as active ingredients or “actives” range in form and function and are outlined in the following paragraphs.

Analgesics: Most commercial topical analgesics use a counter-irritant, such as methyl salicylate, menthol, camphor, eucalyptol and derivatives or mixtures thereof, or rubefacients, such as capsicum, oleoresin chloroform and the like, formulated as an ointment or gel.

The use of counter-irritants and rubefacients to achieve analgesia are well known in the art. Arora, U.S. Pat. No. 5,223,257, discloses an analgesic composition of methyl salicylate, olive oil, eucalyptus oil and isopropyl alcohol. Nichols, U.S. Pat. No. 5,223,267, discloses an analgesic composition of cellulosic powder, counter-irritant (salicylates, menthol, comphor, eucalyptol), analgesic (aspirin, triethanolamine salicylate, ibuprofen), steroid (hydrocortisone), mineral oil, emollient and alcohol. Fisher, U.S. Pat. No. 3,880,996, discloses a preparation of salicylate, menthol, polysiloxane and a vasodilator, such as histamine. Beck, U.S. Pat. No. 5,073,366, discloses a composition containing camphor and eucalyptus oil. Elden, U.S. Pat. No. 5,814,659, discloses a topical composition of a lidocaine analgesic, benzyl alcohol, urea, fatty acid, emulsifier, gel, preservative and organic base. Saitoh et al, U.S. Pat. No. 4,775,667, discloses a topical composition of ethylene glycol monosalicilate, methanol and a small amount of corticosteroid. Hosick, U.S. Pat. No. 4,120,976, discloses the use of methylenedioxyamphetamine to treat arthritis. None of the above cited patents teach or suggest the use of the method and composition outlined in the present invention.

Nonsteroidal Anti-Inflammatory Agents

Nonsteroidal anti-inflammatory agents (NSAIDs) are also useful in relieving pain and tissue swelling, chiefly by inhibiting the biosynthesis of prostaglandins. In small doses, NSAIDs have an analgesic action, but full doses have both analgesic and anti-inflammatory actions, and are effective in reducing pain and swelling. While pain relief from a headache can be obtained with a single 200-400 mg dose of ibuprofen, a full anti-inflammatory effect for bursitis might require 3,200 mg/d of the same drug. NSAIDs fall in seven major classes: proprionic acid derivatives, indole derivatives, fenamates, pyrrolealkanoic acids, pyrazolone derivatives, oxicams and salicylic acids.

Adult Daily Suggested Daily NSAID Oral Dosage Topical Dosage Indomethacin 200 mg 50 mg Sulindac 400 mg 100 mg Tolmetin 1,800 mg 500 mg Piroxicam 20 mg 5 mg Diclofenac potassium 200 mg 50 mg Diclofenac sodium 200 mg 50 mg Fenoprofen 3,200 mg 800 mg Flurbiprofen 300 mg 70 mg Ibuprofen 3,200 mg 800 mg Ketoprofen 300 mg 70 mg Naproxen 1,500 mg 350 mg Etodolac 1,200 mg 300 mg Aspirin 3,600 mg 800 mg Diflunisal 1,500 mg 350 mg

Anti-inflammatory Agents: Inflammation is a fundamental pathologic process involving complex reactions that occur in the affected blood vessels and adjacent tissues in response to an injury or abnormal stimulation caused by a physical, chemical, or biologic agent. The acute inflammatory response begins after cellular injury due to microorganisms, physical agents (such as burns, radiation, and trauma), chemicals, necrotic tissue, and immunological reactions. Five classic signs are manifested in acute inflammation; redness, heat, pain and loss of function. These signs are induced by changes which take place in the microvasculature (arterioles, capillaries, and venules) and the interstitial areas (fluid-filled regions between cells and tissues). These include changes in vascular flow and caliber, changes in vascular permeability, and leucocyte exudation. The first change involves vasodilation of the vessels and increased blood flow. The second change involves increased permeability of the blood vessels with a movement of fluid and proteins out of the vessels creating edema of the tissues. The final change occurs as white blood cells infiltrate and accumulate in the surrounding tissue. The increased blood flow and permeability of the microvascular system at the inflamed body part facilitates treatment to the area by using a penetration enhancer to deliver the bio-affective agents.

The spread of the acute inflammatory response following injury to a small area of tissue suggests that chemical substances are released from injured tissues, spreading outwards into uninjured areas. These chemicals, called endogenous chemical mediators, cause vasodilation, emigration of neutrophils, chemotaxis and increased vascular permeability. Histamine is a chemical mediator in acute inflammation and causes vascular dilatation and vascular permeability. It is stored in mast cells, basophil and eosinophil leucocytes, and platelets. Histamine release is stimulated by complement components C3a and C5a and by lysosomal proteins released from neutrophils. Prostaglandins are a group of long-chain fatty acids derived from arachidonic acid. They increase vascular permeability, and platelet aggregation. Drugs such as aspirin and NSAIDs inhibit one of the enzymes involved in prostaglandin synthesis. Other chemical mediators include; leukotrienes, serotonin and lymphokines. Plasma contains four enzymatic cascade systems; complement, the kinins, the coagulation factors and the fibrinolytic system.

Chronic inflammations are characterized by a longstanding dull pain, and indurated swelling, and the presence of granulation tissue. The predominant cells seen in chronic inflammation are the mononuclear leukocytes, such as macrophages, lymphocytes, and plasma cells. A fibroblastic proliferation is seen more often than a fluid exudate. Some bio-affective agents with anti-inflammatory properties are the following:

CORTICOSTERIODS: Alclometasone dipropionate, Amcinonide, Augmented betamethasone dipropionate, Beclomethasone dipropionate, Betamethasone, Betamethasone benzoate, Betamethasone dipropionate, Betamethasone sodium phosphate, Betamethasone valerate, Clobetasol propionate, Clocortolone pivalate, Cortisone, Desonide, Desoximetasone, Dexamethasone, Dexamethasone acetate, Dexamethasone sodium phosphate, Diflorasone acetonide, Diflorasone diacetate, Flunisolide, Fluocinolone acetonidem, Fluocinonide, Fluocinolone acetonide, Flurandrenolide, Fluticasone propionate, Halcinonide, Halobetasol propionate, Hydrocortisone, Hydrocortisone acetate, Hydrocortisone butyrate, Hydrocortisone sodium phosphate, Hydrocortisone valerate, Methylprednisolone, Methylprednisolone acetate, Methylprednisolone sodium succinate, Mometasone furoate, Prednisolone acetate, Prednisolone sodium phosphate, Prednisolone tebutate, Prednisone, Triamcinolone, Triamcinolone acetonide, Triamcinolone diacetate, Triamcinolone hexacetonide

Methyl-Sulfonyl-Methane: Methyl-sulfonyl-methane (MSM) or dimethyl sulfone is essentially DMSO with an extra oxygen molecule and lacks the lipid-solubility of DMSO, but can be coupled with another penetration enhancer. In the body, MSM gives up its sulfur to form methionine and cysteine for connective tissue. MSM is anti-inflammatory and analgesic and useful for muscle soreness and cramps, prevents cartilage degeneration and improves joint flexibility. The therapeutic dosage range for MSM is 2-10 grams orally per day. The recommended topical dosage range is 1-5 grams.

Numerous patents for MSM were filed by Herschler. U.S. Pat. No. 4,296,130, discloses a method for softening skin; U.S. Pat. No. 4,477,469 discloses a composition of MSM and carbamide to soften skin; U.S. Pat. No. 4,863,748 discloses a method for adding sulfur to the diet with MSM; U.S. Pat. No. 4,973,605 discloses a method for treating muscle cramps associated with arthritis with oral MSM; and U.S. Pat. No. 5,071,878 discloses a method for using MSM in a diet for sulfur and health reasons. None of the above cited patents teach or suggest the use of the method and composition outlined in the present invention.

Zinc Compounds: Osteoporosis is characterized by progressive loss of bone architecture and mineralization leading too the loss of bone strength and an increased fracture rate. The skeleton is constantly being remodeled by a balance between osteoblasts, that lay down new bone, and osteoclasts, that break down or resorb bone.

Zinc plays a physiological role in the regulation of bone metabolism, by stimulating bone formation and mineralization and an inhibitory effect on bone resorption. Zinc activates aminoacyl-tRNA synthetase in osteoblastic cells, stimulates cellular protein synthesis, and inhibits osteoclast-like cell formation in marrow cells. Bone zinc content is decreased by development, with aging, skeletal unloading, and postmenopausal conditions. Zinc plays a role in the preservation of bone mass. Most zinc compounds, such as zinc sulfate, are useful for the prevention of osteoporosis, but a recent study confirmed that .beta.-Alanyl-L-histidinato zinc (AHZ) has a potent effect on bone formation and calcification. Yamaguchi M, Role of Zinc in Bone Formation and Bone Resorption, J. of Trace F. Elements and Experimental Medicine 1998; 11:119-135.

Zinc compounds have anti-inflammatory and anti-infective properties. In a recent published article, Petrus E J et al., Current Therapeutic Research, 1998; 59/9: 595-607, the inventor served as chief investigator for a randomized, double-masked, placebo-controlled clinical study of the effectiveness of zinc acetate lozenges on common cold symptoms in allergy-tested subjects. Those subjects who used the zinc lozenges had both a shorter duration and severity of common cold symptoms. Those subjects who were positive for allergies, were more responsive to zinc by having a shorter duration of nasal symptoms. The study cited many references that reported the following benefits and effects of zinc compounds:

Zinc is an essential trace element in human biology that is known to be necessary for many biologic functions, such as growth, appetite, testicular maturation, skin integrity, mental activity, wound healing, and immune system maintenance. Approximately 300 enzymes are known to require zinc for their activities. Zinc deficiency in humans is widespread and is more prevalent in areas where the population subsists on cereal proteins. Clinical manifestations of zinc deficiency include: growth retardation, hypogonadism in males, neurosensory disorders, cell-mediated immunological dysfunctions, increased maternal morbidity, premature delivery, and adversely affects the proliferation, regulation and maturity of lymphocytes.

Zinc has been shown to be an essential element for the function of the immune system. Regarding the effect of zinc on allergies, it is known that mast cells have been implicated as mediators of Type I allergic reactions. Mast cell derived reactions result from the release of histamine, heparin, prostaglandins, SRS-A, and various vasoactive amines from granules on the surface of mast cells, possibly including kinins. One product of mast cell-induced inflammation is fever. The inhibitory effect of zinc on histamine release from mast cells are attributed to its action on the stabilization of the mast cell membrane. Zinc ions were found to stabilize cell plasma membranes and prevent induced histamine and vasoactive amine release from tissue mast cells. It has been observed that unsequestered zinc ions (4 to 20 millimolar) are released in inflammation from mast cell granules suggesting a common linkage with inflammation. Zinc is a competitive antagonist of the calcium-dependent IgE and f-met peptide mediated histamine release from human basophils and suggested that zinc compounds might be considered for the treatment of autoimmune disorders.

Zinc compounds are acknowledged as anti-inflammatory agents, as astringents and beneficial in wound healing, and have antimicrobial, antifungal and antiviral activity. Zinc is the active agent in formulations to treat diaper rash, decubitus ulcers, and abrasions. Zinc stabilizes the cell membranes and inhibits the formation of free radicals. Zinc also strengthens the integrity of blood vessel walls by reducing the membrane permeability and stopping bleeding. Unlike other metals, zinc is virtually nontoxic.

Aloe Vera Extract: Aloe vera has been well reported to have anti-inflammatory and analgesic properties, but its use in treating musculoskeletal disorders has only recently been described. One study treated patients with a diagnosis of fibromyalgia and/or chronic fatigue syndrome with aloe vera gel extract and found that there was a remarkable reduction in initial symptom severity and continued improvement during the course of the study. Dykman K D, Tone C, Ford C, Dykman R A, The effects of nutritional supplements on the symptoms of fibromyalgia and chronic fatigue syndrome. Interg Physiol Behav Sci 1998 January-March; 33:61-71. Both topical and oral treatments with aloe vera were found to increase the synthesis of glycosaminoglycans and enhance would healing. Chithra P, Sajithlal G B, Chandrakasan G, Influence of Aloe vera on the glycosaminoglycans in the matrix of healing dermal wounds in rats. J Ethnopharmacol 1998 January; 59(3). 179-86. Aloe vera also increased the biosynthesis of collagen. Chithra P, Sajithlal G B, Chandrakasan G, Influence of Aloe vera on collagen turnover in healing of dermal wounds in rats. Indian J Exp Biol 1998 September; 36(9):896-901. Aloe vera mixed with a nitric oxide inhibitor (L-NAME) improved wound healing and prevented dermal ischemia by reversing the effects of thromboxane synthase. Effect of the combination of Aloe vera, nitroglycerin, and L-NAME on wound healing in the rat excisional model. J. Altern Complement Med 1997 Summer; 3(2): 149-53.

The use of Aloe vera is well known in the art. Carpenter et al, U.S. Pat. No. 5,786,342, discloses a method of reducing symptoms associated with chronic respiratory diseases using acetylated mannan from aloe vera. Strickland et al, U.S. Pat. No. 5,824,659, discloses the use of a oligosaccharide from Aloe to inhibit the loss of skin immunocompetency from ultraviolet irradiation.

CHELATING AGENTS: Bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and/or a combination of supportive agents may be added to said composition prior to administration such that said topical preparation further contains at least one of said calcium chelators, bisphosphonates, antibiotics, antimicrobial agents, cytostatic agents, calcium ATPase and pyrophosphatase pump inhibitors, calcium phosphate-crystal dissolving agents, agents effective against calcium phosphate-crystal nucleation and crystal growth, and a combination of supportive agents. Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof.

Antibiotics: Antibiotics may be added to the base formulation in order to impart anti-micorbial attributes. Beta-lactam antibiotics are selected from at least one of penicillin, phenethicillin, ampicillin, aziocillin, bacmpicillin, carbenicillin, cylclacillin, mezlocillin, piperacillin, epicillin, hetacillin, cloxacillin, dicloxacillin, methicillin, nafcillin, oxacillin, and pharmaceutically acceptable salts thereof. Aminoglycoside antibiotics are selected from at least one of streptomycin, kanamycin, gentamycin, amikacin, neomycin, pardomycin, tobramycin, viomycin, and pharmaceutically acceptable salts thereof. Tetracyclines are selected from at least one of tetracycline, chlortetracycline, demeclocycline, doxycycline, methacycline, oxytetracycline, rolitetracycline, minocycline, sancycline and pharmaceutically acceptable salts thereof. Beta-lactam antibiotics, aminoglycoside antibiotics, tetracyclines, trimethoprim and sulpha-trimethoprim combinations, nitrofurantoin, and pharmaceutically acceptable salts thereof, and mixtures thereof.

Anti-fungal Medications: Anti fungal medicines may also be added to the base, including: Clotrimazole, Miconazole, Butenafine, Naftifine, Ketoconazole, Ciclopirox, Terbinafine, Tolnaftate, Undecylenic acid and undecylenate salts (e.g., calcium undecylenate, copper undecylenate, zinc undecylenate), Sulconazole, sertaconazole, Econazole, boric acid, Ciclopirox olamine, Betamethasone,

Accordingly, it is a principal object of the invention to provide a novel and versatile topical composition base that can accept various medicaments and cosmetic ingredients wherein said composition has enhanced skin penetration.

It is another object of the invention to provide a topical composition base that has enhanced spread ability and feel on the skin.

It is another object of the invention to provide a topical composition that is pH optimized to soothe and comfort the skin.

It is another object of the invention to provide a topical composition that is a skin formulation for treating skin disorders.

It is another object of the invention to provide a topical preparation that contains magnesium, EDTA, citric acid, MSM, and sulfate.

It is another object of the invention to provide a topical formulation which includes one or more active ingredients or medicines to be used on the skin.

It is a further object of the invention to provide a skin formulation which is alcohol free.

It is a yet another object of the invention to provide a topical skin treatment for psoriasis, eczema, and dermatitis.

It is yet another object of the invention to provide a topical skin treatment for acne.

It is yet another object of the invention to provide a topical skin treatment containing cosmetically active compositions.

It is yet another object of the invention to provide a skin formulation which does not cause adverse side affects.

It is an object of the invention to provide improved elements and arrangements thereof for the purposes described which is inexpensive, dependable and fully effective in accomplishing its intended purposes.

These and other objects of the present invention will become readily apparent upon further review of the following specification.

### DETAILED DESCRIPTION OF THE PREFERRED EMBODIMENTS

The present invention relates to a formulation for treating and alleviating skin disorders including, but not limited to, dermatitis, rough skin, cracking, itching and psoriasis. Said preparation comprising an novel combination comprising an elevated level of metal chelator (EDTA), dimethyl Sulfone (anti-inflammatory), citric acid) which forms citrate upon dissolution in the aqueous phase, and magnesium sulfate (which releases magnesium in the aqueous phase). The overall effect of the novel combination provides for enhanced penetration through the epidermis and for the delivery of Mg and citrate to the dermal strata. A representative formulation follows:

| | |
|---|---|
| Aloe Barbadensis Leaf Juice Powder or | (between 40-90%) |
| aloe vera leaf water | |
| Butyrospermum Parkii (Shea Butter) | (between 0.20 and 20%) |
| Caprylyl Glycol | (between 0.1 and 2%) |
| Carbomer | (between 0.40 and 2%) |
| Cellulose Gum | (between 0.1 and 5%) |
| Chamomilla Recutita (Matricaria) | (between 0.1 and 4%) |
| Flower Extract | |
| Citric Acid | (between 0.15 and 5%) |
| Dimethicone Copolyol | (between 0.20 and 10%) |
| Dimethyl Sulfone (MSM) | (between 0.20 and 15%) |
| Disodium EDTA | (between 0.10 and 3%) |
| Hexylene Glycol | (between 0.05 and 1.0%) |
| Isopropyl Myristate | (between 0.20 and 10%) |
| Magnesium Sulfate (Epson Salts) | (between 0.10 and 10%) |
| Phenoxyethanol | (between 0.10 and 1%) |
| Polysorbate 80 | (between 0.50 and 3%) |
| Potassium Sorbate | (between 0.2 and 2%) |
| Propylene Glycol | (between 0.20 and 10%) |
| Simmondsia Chinensis (Jojoba) Seed Oil | (between 0.20 and 15%) |
| Tocopherol | (between 0.15 and 2%) |
| Gamma Tocotrienol | (between 0.10 and 1%) |
| Delta Tocotrienol | (between 0.10 and 1%) |
| Triethanolamine | (between 0.25 and 5%) |
| Water | (between 40 and 85%) |

In one embodiment, the formulation can be prepared as a lotion or as a gel or viscous cream and includes the steps, generally:

Part A
Disperse the EDTA in the water with vigorous mixing
Add the Carbopol Ultrez 10 NF in the water/EDTA by pouring the powder onto the water, and allowing it to completely wet (approx. 10-20 minutes).
After the polymer is wetted out, mix the polymer at approximately 400-600 RPM to get a smooth dispersion, approximately 20 minutes
Add the Isopropyl Myristate to the dispersion with stirring
Add the Aloe and CMC

Part B
Gently warm the water to approximately 35° C. and disperse the Methyl Sulfonyl Methane in the water with vigorous mixing to complete dissolution.
Add the Magnesium Sulfate with vigorous mixing to complete dissolution.
Add the Citric Acid with vigorous mixing to complete dissolution.
Add the EDTA with vigorous mixing to complete dissolution.
Add CAP 5

Part C
Disperse the hydrocortisone in the Propylene Glycol. Mix to homogeneous suspension. Add Polysorbate 80 with vigorous mixing.
Add the rest of ingredients of Part C in the order listed.

COMBINE PARTS A & B with vigorous mixing

Part E
Add 10× Aloe Gel to the water with stirring
Add Sodium Carboxy Methyl Cellulose to the Aloe/Water solution with 400-600 RPM mixing
Add Part E to Part A/B/C/D
Add Part F to Part A/B/C/D/E

In another embodiment, the formulation can be prepared as follows:

Part A
Dissolve EDTA in water
Add Carbopol Ultrez 10 NF and allow to wet for approximately 10 minutes (until completely wetted)
Mix at medium shear for approximately 15 minutes
Add/Dissolve MSM
Add/Dissolve Magnesium Sulfate
Add/Dissolve the Citric Acid
Add IPM, mix
Add Aloe Powder
Add Jeen CAP 5 (preservative)
Add CEKOL with high shear mixing until completely LUMP free

Part B
Add hydrocortisone (or other active) to propylene glycol and homogenize . . . extremely HIGH sheer
Add Polysorbate 80 with vigorous mixing.
Add the rest of ingredients of Part B in the order listed.

COMBINE PARTS A & B with vigorous mixing

Part C
Add TEA with vigorous mixing.

The active ingredients may be added into either the water or the oil phase. In one embodiment, hydrocortisone is added to the oil phase at a level of 0.25% by weight. In another embodiment, hydrocortisone is added at 0.5% by weight. In a more preferred embodiment, hydrocortisone is added in the oil phase at 1.0%.

For a more complete understanding of the present improved composition, reference is made to the following examples. The following examples are illustrative of the present improved composition and are not intended in any way as a limitation upon the scope thereof.

### Example 1

### Hydrocortisone Gel

An appropriate amount of purified water is measured into a container and gently heated to approximately 40 C. Carbopol Ultrez 10 is added to the water and allowed to wet for approximately 20 minutes followed by low shear mixing for approximately 20 minutes. Aloe powder is added followed disodium EDTA, Magnesium sulfate, Citric acid, and methyl sulfonyl methane. The solution is mixed until complete solubility of all components. Subsequently, isopropyl myristate and preservative is added. Finally, a modified corn starch product called CEKOL is added with high shear in order to obtain a slightly viscous aqueous phase.

In another container, propylene glycol is measured in. Hydrocortisone or hydrocortisone acetate is added directly to the propylene glycol with high shear mixing. After obtaining a complete suspension, the emulsifier is added (Polysorbate 80) followed by the addition of silicone, shea butter, chamomile, and vitamin E. Other oil soluble ingredients may be added herein such as Mahonia aquifolium, red palm oil or fractions thereof including tocotrienols alpha, beta, gamma, or delta, nobilitin, or synthetics such as polyethylene glycol or long chain fatty acids.

The aqueous and oil phases are then combined by slowly adding the aqueous phase to the oil phase with moderate shear mixing.

### Example 2

### Hydrocortisone, Neomycin, and Bacitracin Gel

Hydrocortisone gel: An appropriate amount of purified water is measured into a container and gently heated to approximately 40 C. Carbopol Ultrez 10 is added to the water and allowed to wet for approximately 20 minutes followed by low shear mixing for approximately 20 minutes. Aloe powder is added followed disodium EDTA, Magnesium sulfate, Citric acid, and methyl sulfonyl methane. The solution is mixed until complete solubility of all components. Then the Neomycin and bacitracin are added and mixed to complete dissolution. Subsequently, isopropyl myristate and preservative is added. Finally, a modified corn starch product called CEKOL is added with high shear in order to obtain a slightly viscous aqueous phase.

In another container, propylene glycol is measured in. Hydrocortisone or hydrocortisone acetate is added directly to the propylene glycol with high shear mixing. After obtaining a complete suspension, the emulsifier is added (Polysorbate 80) followed by the addition of silicone, shea butter, chamomile, and vitamin E. Other oil soluble ingredients may be added herein such as Mahonia aquifolium, red palm oil or fractions thereof including tocotrienols alpha, beta, gamma, or delta, nobilitin, or synthetics such as polyethylene glycol or long chain fatty acids.

The aqueous and oil phases are then combined by slowly adding the aqueous phase to the oil phase with moderate shear mixing.

### Example 3

### Salicylic Acid Gel

Hydrocortisone gel: An appropriate amount of purified water is measured into a container and gently heated to approximately 40 C. Carbopol Ultrez 10 is added to the water and allowed to wet for approximately 20 minutes followed by low shear mixing for approximately 20 minutes. Aloe powder is added followed disodium EDTA, Magnesium sulfate, Citric acid, and methyl sulfonyl methane. The solution is mixed until complete solubility of all components. Then salicylic acid is added with high shear mixing. Subsequently, isopropyl myristate and preservative is added. Finally, a modified corn starch product called CEKOL is added with high shear in order to obtain a slightly viscous aqueous phase.

In another container, propylene glycol is measured in. The, the emulsifier is added (Polysorbate 80) followed by the addition of silicone, Shea butter, chamomile, and vitamin E. Other oil soluble ingredients may be added herein such as Mahonia aquifolium, red palm oil or fractions thereof including tocotrienols alpha, beta, gamma, or delta, nobilitin, or synthetics such as polyethylene glycol or long chain fatty acids.

The aqueous and oil phases are then combined by slowly adding the aqueous phase to the oil phase with moderate shear mixing.

### Example 4

### Hydrocortisone and Tetracycline Lotion

Hydrocortisone gel: An appropriate amount of purified water is measured into a container and gently heated to approximately 40 C. Carbopol Ultrez 10 is added to the water and allowed to wet for approximately 20 minutes followed by low shear mixing for approximately 20 minutes. Aloe powder is added followed disodium EDTA, Magnesium sulfate, Citric acid, and methyl sulfonyl methane. The solution is mixed until complete solubility of all components. Then tetracycline is added and mixed to complete solution. Subsequently, isopropyl myristate and preservative is added. Finally, a modified corn starch product called CEKOL is added with high shear in order to obtain a slightly viscous aqueous phase.

In another container, propylene glycol is measured in. Hydrocortisone or hydrocortisone acetate is added directly to the propylene glycol with high shear mixing. After obtaining a complete suspension, the emulsifier is added (Polysorbate 80) followed by the addition of silicone, Shea butter, chamomile, and vitamin E. Other oil soluble ingredients may be added herein such as Mahonia aquifolium, red palm oil or fractions thereof including tocotrienols alpha, beta, gamma, or delta, nobilitin, or synthetics such as polyethylene glycol or long chain fatty acids.

The aqueous and oil phases are then combined by slowly adding the aqueous phase to the oil phase with moderate shear mixing.

Accordingly, it is a principal object of the invention to provide a versatile and functional topical base with enhanced lubricity, optimal pH, elevated levels of chelating agents, elevated levels of citrates, magnesium, and sulfate. Still another object of the invention is to provide topical base composition with enhanced penetration. It is a further object of the invention to provide such a topical base to used with various medicaments and active ingredients to the treatment of skin conditions.

It is to be understood that the present invention is not limited to the methods described above, but encompasses any and all methods within the scope of the following claims.

### REFERENCES

Koo J. The psychosocial impact of acne: Patient's perceptions. J Am Acad Dermatol. 1995; 32:S26-S30.
Sulzberger M B, Zaidems S H. Psychogenic factors in dermatological disorders. Med Clinicians North Am. 1948; 32:669.
Cunliffe W J. Acne and unemployment. Br J Dermatol. 1986; 115:386.
Goulden V, Stables G I, Cunliffe W J. Prevalence of facial acne in adults. J Am Acad Dermatol. 1999; 41:577-580.
Thiboutot D M. New treatments and therapeutic strategies for acne. Arch Fam Med. 2000; 9:179-187.
Leyden J J. New understandings of the pathogenesis of acne. J Am Acad Dermatol. 1995; 32:S15-S25.
Taylor S C, Cook-Bolden F, Rachman Z, Strachan D. Acne vulgaris in skin of color. J Am Acad Dermatol. 2002; 46:S98-S106
Eady E A, Bojar R A, Jones C E, Cove J H, Holland K T, Cunliffe W J. The effects of acne treatment with a combination of benzoyl peroxide and erythromycin on skin carriage of erythromycin-resistant propionibacteria. Br J Dermatol. 1996; 134:107-113.
Leyden J, Levy S. The development of antibiotic resistance to propionibacterium acnes. Cutis. 2001; 67:21-24.
Wolf J E Jr. Potential anti-inflammatory effects of topical retinoids and retinoid analogues. Adv Ther. 2002; 19:109-118.
Shalita A. The integral role of topical and oral retinoids in the early treatment of acne. J Eur Acad Dermatol Venereol. 2001; 15:43-49.
Quigley J W, Bucks D A. Reduced skin irritation with tretinoin containing polyolprepolymer-2, a new topical tretinoin delivery system: a summary of preclinical and clinical investigations. J Am Acad Dermatol. 1998; 38:S5-S10.
Millikan L E. Adapalene: an update on newer comparative studies between the various retinoids. Int J Dermatol. 2000; 39:784-788.
Shalita A, Weiss J S, Chalker D K. A comparison of the efficacy and safety of adapalene gel 0.1% and tretinoin gel 0.025% in the treatment of acne vulgaris: a multicenter trial. J Am Acad Dermatol. 1996; 34:482-485.
Cunliffe W J, Danby F W, Dunlap F, et al. Randomised, controlled trial of the efficacy and safety of adapalene gel 0.1% and tretinoin cream 0.05% in patients with acne vulgarism Eur J Dermatol. 2002; 12:350-354.
Galvin S A, Gilbert R, Baker M, Guibal F, Tuley M R. Comparative tolerance of adapalene 0.1% gel and six different tretinoin formulations. Br J Dermatol. 1998; 52:34-40.
Webster G F, Berson D, Stein L F, et al. Efficacy and tolerability of once-daily tazarotene 0.1% gel versus once-daily tretinoin 0.025% gel in the treatment of facial acne vulgaris: a randomized trial. Cutis. 2001; 67:4-9.
Leyden J J, Tanghetti E A, Miller B, Ung M, Berson D, Lee J. Once-daily tazarotene 0.1% gel versus once-daily tretinoin 0.1% microsponge gel for the treatment of facial acne vulgaris: a double-blind randomized trial. Cutis. 2002; 69:9-12.
Webster G F, Guenther L, Poulin Y P, et al. A multicenter, double-blind, randomized comparison study of the efficacy and tolerability of once-daily tazarotene 0.1% gel and adapalene 0.1% gel for the treatment of facial acne vulgarism Cutis. 2002; 69:4-11.
Graupe K, Cunliffe W J, Gollnick H P, Zaumseil R P. Efficacy and safety of topical azelaic acid (20 percent cream): an overview of results from European clinical trials and experimental reports. Cutis. 1996; 57(1 suppl):20-35.
Yamaguchi M, Role of Zinc in Bone Formation and Bone Resorption, J. of Trace F. Elements and Experimental Medicine 1998; 11:119-135.
Petrus E J et al., Current Therapeutic Research, 1998; 59/9: 595-607

What is claimed is:

1. A formulation for a topical skin treatment that contains EDTA, Dimethyl Sulfone, citric acid, and magnesium sulfate within a stable emulsion wherein said EDTA is present at between 0.10% to 3% wt/wt %; said Dimethyl Sulfone is present at between 0.20% and 15% wt/wt %; said citric acid is present between 0.15% and 5% wt/wt %; and said Magnesium Sulfate is present at between 0.10 and 10% wt/wt %, collectively contained within a topical formulation.

2. The combination of ingredients of claim 1 wherein said combination provides for enhanced penetration through the epidermis and delivery of magnesium and citrate ions into the dermis and hypodermis.

3. The combination of ingredients of claim 1 wherein said combination provides for the delivery of magnesium and citrate ions to the dermis and hypodermis to effective treat skin conditions.

4. The skin conditions of claim 3 that include psoriasis, dermatitis, eczema, and acne vulgaris.

5. The combination of the ingredients of claim 1 wherein said topical formulation is comprised of standard emollients, carrier oils binders, polymers, skin conditioners, skin protectants, oils, carriers, emulsifiers, preservatives, antioxidants, lubricating agents, and water in an oil in water emulsion.

6. The formulation of claim 5 wherein said topical formulation ingredients are comprised of Aloe Barbadensis, Butyrospermum Parkii (Shea Butter), Caprylyl Glycol, Carbomer, Cellulose Gum, Chamomilla Recutita (Matricaria) Flower Extract, Dimethicone Copolyol, Hexylene Glycol, Isopropyl Myristate, Phenoxyethanol Polysorbate 80, Potassium Sorbate, Propylene Glycol, Simmondsia Chinensis (Jojoba) Seed Oil, Tocopherol, Triethanolamine, Water.

7. The formulation of claim 1 wherein said formulation acts as enhanced base carrier for an antibiotic.

8. The antibiotics of claim 7 wherein said antibiotics are selected from the groups consisting of .beta.-lactam antibiotics, aminoglycoside antibiotics, tetracycline antibiotics, trimethoprim antibiotics, nitrofurantoin antibiotics and pharmaceutically acceptable salts thereof, and mixtures thereof.

9. The formulation of claim 1 wherein said formulation acts as a topical carrier formulation for an analgesic.

10. The analgesics of claim 9 wherein said analgesics are selected from the group consisting of methyl salicylate, menthol, camphor, eucalyptol, capsicum, oleoresin, chloroform, and derivatives and mixtures thereof.

11. The formulation of claim 1 wherein said formulation acts as a topical carrier formulation for a corticosteroids.

12. The corticosteroid of claim 11 wherein said corticosteroid is selected from the groups consisting of alclonetasone dipropionate, amcinonide, beclomethasone dipropionate, betamethasone, betamethasone 17-valerate, betamethasone 17,21-divalerate, betamethasone 21-acetate, betamethasone 21-butyrate, betamethasone 21-propionate, betamethasone 21-valerate, betamethasone benzoate, betamethasone dipropionate, betamethasone valerate, budesonide, clobetasol propionate, clobetasone butyrate, cortexolone, corticosterone, cortisone, cortisone 17-acetate, 21-deoxybetamethasone, 21-deoxybetamethasone 17-propionate, deoxycorticosterone, desonide, desoxymethasone, dexamethasone, diflorasone diacetate, diflucortolone valerate, fluclorolone acetonide, flumethasone pivalate, fluoconolone acetonide, fluocinonide, fluocortin butyl, fluocortolone, 9-alpha-fluorocortisone, 9-alpha-fluorohydrocortisone, 9-alpha-fluoroprednisolone, fluprednidene acetate, flurandrenolone, halcinonide, hydrocortisone, hydrocortisone 17-acetate, hydrocortisone 17-butyrate, hydrocortisone 17-propionate, hydro cortisone 17-valerate, hydrocortisone 21-acetate, hydrocortisone 21-butyrate, hydrocortisone 21-propionate, hydrocortisone 21-valerate, 17-alpha-hydroxyprogesterone, methylprednisolone acetate, mometasone furoate, prednisolone, prednisone, prednisone 17-acetate, prednisone 17-valerate, progesterone, triamcinolone, and trimcinolone acetonide. or acceptable salts thereof.

13. The formulation of claim 1 wherein said formulation acts as a topical carrier formulation for a nonsteroidal anti-inflammatory drug (NSAID).

14. The NSAIDs of claim 13 wherein; said NSAIDs are selected from the group consisting of Indomethacin, Sulindac, Tolmetin, Piroxicam, Diclofenac potassium, Diclofenac sodium, Fenoprofen, Flurbiprofen, Ibuprofen, Ketoprofen, Naproxen, Etodolac, Aspirin, and Diflunisal and appropriate salts and mixtures thereof.

15. The formulation of claim 1 wherein said formulation acts as a topical carrier formulation for a zinc compound.

16. The zinc compounds of claim 15 wherein; said zinc compounds are selected from the group consisting of zinc oxide, zinc sulfate, and acceptable salts thereof, and mixtures thereof.

17. The formulation of claim 1 wherein said formulation acts as a topical carrier formulation for an anti-fungal agent.

18. The antifungal agents of claim 17 wherein; said antifungal agents are selected from the groups consisting of Clotrimazole, Miconazole, Butenafine, Naftifine, Ketoconazole, Ciclopirox, Terbinafine, Tolnaftate, Undecylenic acid and undecylenate salts (e.g., calcium undecylenate, copper undecylenate, zinc undecylenate), Sulconazole, sertaconazole, Econazole, boric acid, Ciclopirox olamine, Betamethasone, and acceptable salts or mixtures thereof.

19. The formulation of claim 1 wherein said formulation acts as a topical carrier formulation for coal tar.

20. The formulation of claim 1 wherein said formulation acts as a topical carrier formulation for salicylic acid.

21. The formulation of claim 1 wherein said formulation acts as a topical for at least one of an antibiotic, corticosteroid, non-steroidal antiinflammatory drug, zinc compound, analgesic compound or anti-fungal compound.

22. The composition of claim 1 wherein said formulation has a pH less between 5.00 and 5.50.

23. The composition of claim 1 wherein said formulation has a viscosity of between 12,000 and 13,000 centipoises

24. The composition of claim 1 wherein said formulation has a specific gravity of between 1.00 and 1.05.

25. The composition of claim 1 wherein said formulation has a specific gravity of 1.02.

26. The composition of claim 1 wherein said blend of EDTA, Dimethyl Sulfone, citric acid, and magnesium sulfate provides for enhanced penetration through the epidermis.

27. The composition of claim 1 wherein said blend of EDTA, Dimethyl Sulfone, citric acid, and magnesium sulfate provides for the delivery of magnesium cations and citrate anions to the dermis and hypodermis.

28. The composition of claim 1 wherein said topical preparation is applied to the body of a mammal to ease the suffering as caused by or to treat psoriasis including; plaque psoriasis, guttate psoriasis, inverse psoriasis, pustular psoriasis, and erythrodermic psoriasis.

29. The composition of claim 1 wherein said topical is applied to the body of a mammal to ease the suffering due to or treat eczema or dermatitis, including atopic eczema, varicose eczema, discoid eczema, allergic contact dermatitis, irritant contact dermatitis, infantile seborrheic dermatitis, adult seborrheic dermatitis, atopic dermatitis, seborrheic dermatitis.

30. The composition of claim 1 wherein said topical formulation is applied to the body of a mammal to treat acne vulgaris, rosacea, scleroderma, skin stones, fungal infections, bacterial infections, or other skin disorders and diseases with improved efficacy and penetration.

31. The composition of claim 1 wherein said topical formulation is applied to the body of a mammal to treat itching as caused by irritation, bug bites, botanical irritants, poison oak, sumac.
